(19) United States
(12) Patent Application Publication
(10) Pub. No.: US 2016/0222054 A1
(43) Pub. Date: Aug. 4, 2016

(54) PH SWITCHABLE REAGENTS AND METHODS FOR THEIR USE

(71) Applicants: IMPOSSIBLE FOODS INC., Redwood City, CA (US); THE BOARD OF TRUSTEES OF THE LELAND STANFORD JUNIOR UNIVERSITY, Palo Alto, CA (US)

(72) Inventors: Patrick O'Reilly Brown, Redwood City, CA (US); Onorato Campopiano, Hayward, CA (US); R. Edward Watts, Palo Alto, CA (US); Pehr Harbury, Portola Valley, CA (US)

(21) Appl. No.: 15/023,054

(22) PCT Filed: Sep. 19, 2014
(86) PCT No.: PCT/US14/56568

§ 371 Date: Mar. 18, 2016

Related U.S. Application Data
(60) Provisional application No. 61/879,977, filed on Sep. 19, 2013.

(51) Int. Cl. C07K 1/22; C07K 14/415; C12N 9/88; C07D 251/54

(52) U.S. Cl. CPC C07K 1/22; C07D 251/54; C07K 14/415; C12N 9/88; A23L 1/305; C12Y 401/01039; A23V 2002/00

(57) ABSTRACT

This disclosure provides materials and methods for synthesis and use of pH switchable ligands.

### CROSS-REFERENCE TO RELATED APPLICATIONS

This application claims benefit of priority from U.S. Provisional Application Ser. No. 61/879,977, filed on Sep. 19, 2013.

### BACKGROUND

Proteins and other molecules with potential commercial value (e.g., as food ingredients, industrial enzymes, structural materials, or pharmaceuticals) can be found or produced in commercially useful quantities in natural sources. Purification of such molecules from natural sources can be important for optimal utility of the molecules. Methods of purification can be specific for particular molecules, or they can be general for a variety of molecules.

### SUMMARY

This document discloses materials and methods for isolating one or more target molecules (e.g., proteins) using a switchable reagent that binds the target under certain conditions, but does not bind the target or binds the target with a lower affinity under other conditions. The materials and methods provided herein can be useful for efficiently and economically purifying proteins and other molecules from crude solutions. For example, the materials and methods provided herein can be used to purify quantities of particular proteins without the need to produce recombinant, tagged versions of the proteins.

In one aspect, this document features a compound comprising the structure of Formula (III):

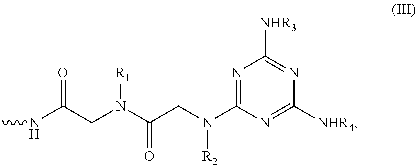

where the N—R1 to N—R4 groups of Formula (III) correspond, respectively, to the R1-R4 groups listed in Tables 1-5, the N of each N—R group in Formula (III) is the N of an R group in the tables, and the wavy line indicates the point of attachment to a substrate, optionally through a linker. The compound can bind a target at a first pH, and not bind the target or bind the target with a lower affinity at a second pH.

In some cases, the N—R1 to N—R4 groups of Formula (III) can correspond, respectively, to the R1-R4 groups listed in Table 2, where the N of each N—R group in Formula (III) is the N of an R group in Table 2. The compound can bind rubisco at a pH of about 6, and release rubisco or bind rubisco with a lower affinity at a pH of about 9.

In some cases, the N—R1 to N—R4 groups of Formula (III) can correspond, respectively, to the R1-R4 groups listed in Table 3, where the N of each N—R group in Formula (III) is the N of an R group in Table 3. The compound can bind rubisco at a pH of about 9, and release rubisco or bind rubisco with a lower affinity at a pH of about 6.

In some cases, the N—R1 to N—R4 groups of Formula (III) can correspond, respectively, to the R1-R4 groups listed in Table 4, where the N of each N—R group in Formula (III) is the N of an R group in Table 4. The compound can bind leghemoglobin at a pH of about 6, and release leghemoglobin or bind leghemoglobin with a lower affinity at a pH of about 9.

In some cases, the N—R1 to N—R4 groups of Formula (III) can correspond, respectively, to the R1-R4 groups listed in Table 5, where the N of each N—R group in Formula (III) is the N of an R group in Table 5. The compound can bind leghemoglobin at a pH of about 9, and release leghemoglobin or bind leghemoglobin with a lower affinity at a pH of about 6.

In some cases, N—R1 can be 3-fluoro-(2-phenyl ethanamine), N—R2 can be 2-amino-N-cyclopropylacetamide, N—R3 can be 2-methyl butylamine, and N—R4 can be (4-bromophenyl) methanamine.

In some cases, N—R1 can be 2-methoxy-ethylamine, N—R2 can be 2-(3-fluorophenyl) ethanamine, N—R3 can be 4-aminobutan-1-ol, and N—R4 can be 2-amino-1-phenylethanol.

The compound can be conjugated to the substrate, optionally through a linker. The substrate can include agarose, sepharose, polystyrene, styrene, iron oxide, magnetic, or paramagnetic beads (e.g., sepharose beads). The linker can include a diamine. For example, the linker can be 4,7,10-trioxa-1,13-tridecanediamine. The substrate can be a component of a chromatography resin.

In another aspect, this document features a method for isolating a target protein. The method can include (a) applying a composition comprising the target protein to a substrate, where the substrate includes a compound comprising the structure of Formula (III)

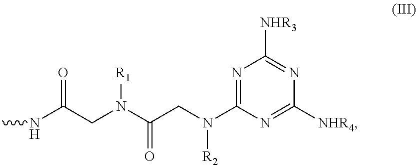

where the N—R1 to N—R4 groups of Formula (III) correspond, respectively, to the R1-R4 groups listed in Tables 1-5, the N of each N—R group in Formula (III) is the N of an R group in the tables, and the wavy line indicates the point of attachment to the remainder of the substrate, optionally through a linker; (b) adjusting the pH of the solution to a second pH, where at the second pH the target protein does not bind the compound or binds the compound with lower affinity, and is eluted from the compound; and (c) collecting the target protein that eluted from the compound. The compound can bind a target at a first pH, and not bind the target or bind the target with a lower affinity at a second pH.

In some cases, the N—R1 to N—R4 groups of Formula (III) can correspond, respectively, to the R1-R4 groups listed in Table 2, where the N of each N—R group in Formula (III) is the N of an R group in Table 2. The compound can bind rubisco at a pH of about 6, and release rubisco or bind rubisco with a lower affinity at a pH of about 9.

In some cases, the N—R1 to N—R4 groups of Formula (III) can correspond, respectively, to the R1-R4 groups listed in Table 3, where the N of each N—R group in Formula (III) is the N of an R group in Table 3. The compound can bind rubisco at a pH of about 9, and release rubisco or bind rubisco with a lower affinity at a pH of about 6.

In some cases, the N—R1 to N—R4 groups of Formula (III) can correspond, respectively, to the R1-R4 groups listed in Table 4, where the N of each N—R group in Formula (III) is the N of an R group in Table 4. The compound can bind leghemoglobin at a pH of about 6, and release leghemoglobin or bind leghemoglobin with a lower affinity at a pH of about 9.

In some cases, the N—R1 to N—R4 groups of Formula (III) can correspond, respectively, to the R1-R4 groups listed in Table 5, where the N of each N—R group in Formula (III) is the N of an R group in Table 5. The compound can bind leghemoglobin at a pH of about 9, and release leghemoglobin or bind leghemoglobin with a lower affinity at a pH of about 6.

In some cases, N—R1 can be 3-fluoro-(2-phenyl ethanamine), N—R2 can be 2-amino-N-cyclopropylacetamide, N—R3 can be 2-methyl butylamine, and N—R4 can be (4-bromophenyl) methanamine.

In some cases, N—R1 can be 2-methoxy-ethylamine, N—R2 can be 2-(3-fluorophenyl) ethanamine, N—R3 can be 4-aminobutan-1-ol, and N—R4 can be 2-amino-1-phenylethanol.

The compound can be conjugated to the substrate, optionally through a linker. The substrate can include agarose, sepharose, polystyrene, styrene, iron oxide, magnetic, or paramagnetic beads (e.g., sepharose beads). The linker can include a diamine. For example, the linker can be 4,7,10-trioxa-1,13-tridecanediamine. The substrate can be a component of a chromatography resin.

In another aspect, this document features a consumable product comprising a target protein and a compound comprising the structure of Formula (III)

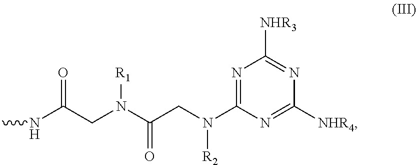

where the N—R1 to N—R4 groups of Formula (III) correspond, respectively, to the R1-R4 groups listed in Tables 1-5, the N of each N—R group in Formula (III) is the N of an R group in the tables, and the wavy line indicates the point of attachment to a substrate, optionally through a linker. The compound can be present at a concentration of less than 1,000 parts per million target protein. The consumable product can be substantially free of chlorophylls, chlorins, metalloids, transition metals, celluloses, or complex polysaccharides. The consumable product can be food-safe.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is an illustration of an exemplary embodiment of methods for solution synthesis of the compounds provided herein.

FIGS. 2A and 2B are a pair of graphs plotting the binding affinity of pH switchable ligands for rubisco. FIG. 2A, candidate compound 16; FIG. 2B, candidate compound 1.

### DETAILED DESCRIPTION

Methods for purifying one or more targets (e.g., proteins from crude solutions) can utilize switchable affinity reagents that have higher affinity for a target under a first set of conditions and lower affinity for the target under a second set of conditions. Conditions that can be used to regulate the affinity of a switchable affinity reagent for a target can include pH, temperature, salinity or ionic strength, and/or the concentration of a metal ion or other reagent. For example, the affinity of a switchable affinity reagent can be controlled by relying on pH dependent changes in charge of residues or chemical moieties on the target, the switchable affinity reagent, or both. The affected residues or chemical moieties can be located at the interface between the affinity reagent and the target. In some embodiments, the affected residues or chemical moieties have a charge that affects the conformation of the affinity reagent, the target, or both. Switchable affinity reagents can include a small molecule compound, and in some embodiments, can further include a nucleic acid sequence that encodes a program for the synthesis of the small molecule compound. Switchable affinity reagents also can be conjugated to a solid or semi-solid support substrate (e.g., a bead).

A switchable affinity reagent (also referred to as a “switchable affinity ligand”) can include a molecule or small molecule compound that is synthesized from smaller subunits, which optionally can include a common parent structure (e.g., a peptide or peptidomimetic backbone, such as an L-peptide backbone, a D-peptide backbone, a 3-peptide backbone, or a peptoid backbone, or a ring structure, such as a benzene ring, phenol ring, toluene, napthalene, cyclohexyl, sugar, aniline, biphenyl structure, pyridine, or triazine). In general, a switchable affinity reagent can contain between about one and about 100 subunits. For example, a switchable affinity reagent can include about one, two, three, four, five, six, seven, eight, nine, ten, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 subunits, or about one to about 20 subunits, about two to about ten subunits, or about two to about five subunits.

The common parent structure of a switchable affinity reagent also can contain one or more (e.g., two, three, four, five, or more) functional moieties that can be used to link the subunits together. In addition, the subunits can have one or more side chains or ring-structure modifications (collectively called “R groups”), each of which can take a variety of chemical forms. R groups can vary in terms of chain length, ring size or number, and/or patterns of substitution, and can include naturally occurring side chains such as the side chains found in L-peptides, proteins, or amino acids, as well as side chains or groups that are not naturally occurring.

Switchable affinity reagents can have a first binding affinity for a target (e.g., a particular protein) under a first set of conditions (also referred to as “binding conditions”), and a second binding affinity for the target under a second set of conditions (also referred to as “release conditions”). The first binding affinity can be stronger than the second binding affinity. For example, the first binding affinity can be at least about two, three, four, five, six, seven, eight, nine, ten, 15, 20, 25, 30, 40, 50, 60, 70, 80, 90, 100, 150, 200, 300, 400, 500, 600, 700, 800, 900, 1000, 2000, 3000, 4000, 5000, 6000, 7000, 8000, 9000, or 10000 times stronger than the second binding affinity.

The first binding affinity and the second binding affinity can be expressed as dissociation constants (Kd). Dissociation constants can have molar units, and a lower dissociation constant can mean that a switchable affinity reagent and a target bind with a higher affinity. Conversely, a higher dissociation constant can be associated with lower binding affinity. In some embodiments, the first binding affinity between a switchable affinity reagent and a target can be a Kd of less than about 2 mM (e.g., less than about 2 mM, 1000 μM, 500 μM, 250 μM, 100 μM, 75 μM, 50 μM, 25 μM, 10 μM, 5 μM, 1 μM, 500 nM, 250 nM, 100 nM, 75 nM, 50 nM, 25 nM, 10 nM, 5 nM, 1 nM, or lower). The second binding affinity can be a Kd that is at least about two times higher than the Kd for the first binding affinity (e.g., at least about two, three, four, five, six, seven, eight, nine, ten, 15, 20, 25, 30, 40, 50, 60, 70, 80, 90, 100, 150, 200, 300, 400, 500, 600, 700, 800, 900, 1000, 2000, 3000, 4000, 5000, 6000, 7000, 8000, 9000, or 10000 times higher than the Kd for the first binding affinity).

In some embodiments, a switchable affinity reagent can be a pH sensitive switchable reagent (also referred to as a “pH switchable reagent” or a “pH switchable ligand”). pH sensitive switchable reagents can bind a target (e.g., a target protein) at a first (“binding”) pH, and can release the target or bind to the target with lower affinity at a second (“release”) pH. The binding pH can be a pH between about 1 and 14 (e.g., about 1, 2, 3, 4, 5, 6, 7, 8, 9, 10, 11, 12, 13, or 14, or any included decimal). The release pH can be a pH between about 1 and 14 (e.g., about 1, 2, 3, 4, 5, 6, 7, 8, 9, 10, 11, 12, 13, or 14, or any included decimal). The binding pH and the release pH can be separated by between about 1 and about 13 pH units. For example, the binding pH and the release pH can be separated by about 1, 2, 3, 4, 5, 6, 7, 8, 9, 10, 11, 12, or 13 pH units.

In some embodiments, a target can bind to a pH switchable reagent in acidic conditions, and can be released from the pH switchable reagent in basic conditions. For example, a target can bind to a pH switchable reagent at a pH of about 1, 1.5, 2, 2.5, 3, 3.5, 4, 4.5, 5, 5.5, 6, or 6.5, or a pH between about 1 and about 5, between about 2 and about 6, or between about 3 and about 7, and can be released from the pH switchable reagent at a pH of about 7.5, 8, 8.5, 9, 9.5, 10, 10.5, 11, 11.5, 12, 12.5, 13, 13.5, or 14, or a pH between about 7 and about 11, between about 8 and about 12, between about 9 and about 13, or between about 10 and about 14. In some embodiments, for example, the binding pH can be about 6, and the release pH can be about 9.

In some embodiments, a target can bind to a pH switchable reagent in basic conditions, and can be released from the pH switchable reagent in acidic conditions. For example, a target can bind to a pH switchable reagent at a pH of about 7.5, 8, 8.5, 9, 9.5, 10, 10.5, 11, 11.5, 12, 12.5, 13, 13.5, or 14, or a pH between about 7 and about 11, between about 8 and about 12, between about 9 and about 13, or between about 10 and about 14, and can be released from the pH switchable reagent at a pH of about 1, 1.5, 2, 2.5, 3, 3.5, 4, 4.5, 5, 5.5, 6, or 6.5, or a pH between about 1 and about 5, between about 2 and about 6, or between about 3 and about 7. In some embodiments, for example, the binding pH can be about 9 and the release pH can be about 6.

pH sensitive switchable affinity reagents can have a pKa between about 1 and 14 (e.g., about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or 14, or about 1 to about 5, about 2 to about 6, about 3 to about 7, about 4 to about 8, about 5 to about 9, about 6 to about 10, about 7 to about 11, about 8 to about 12, about 9 to about 13, or about 10 to about 14). For example, a pH sensitive switchable affinity reagent can have a pKa of about 7, and can be neutrally charged at a pH of about 9 and positively charged at a pH of about 5.

The pH switchable reagents provided herein can be used to purify target molecules (e.g., proteins) from a mixture. Target molecules can have, for example, industrial value or clinical or environmental effects. In some cases, a target molecule can be a protein for use as a food ingredient. Target molecules also can be enzymes (e.g., industrial enzymes), or can be used as therapeutic agents. For example, a target protein can be an antibody or other protein that can be used to treat a disease or disorder (e.g., cancer). Target molecules also can include analytes for diagnostic tests or other clinical uses. In some embodiments, a target molecule can be used in the production of a consumable product, such as a meat substitute food product or another food product for human or animal consumption.

A target molecule also can be a molecule whose presence in a mixture is undesirable. For example, a target can be a toxin or an allergen, a molecule that contributes to an undesirable taste, odor, or visual appearance in a product, or a molecule whose activity decreases the stability of a desired material. An example of such a target protein is hordein in barley; the removal of hordein from a fermented barley beverage would render the beverage “gluten free.” Another example of such a target protein is RNAse in a cell extract, where RNA is the desired material.

In some embodiments, a pH switchable reagent as provided herein can be used to purify a target molecule (e.g., a protein) from a mixture that is or is derived from a cellular lysate from an animal or non-animal source. Non-animal sources include, without limitation, plants, fungi, bacteria, yeast, algae, archaea, and genetically modified organisms such as genetically modified bacteria or yeast. In some cases, a target molecule can be produced by chemical or in vitro synthesis.

When the one or more target proteins are be derived from plant sources, any suitable plant source can be used, including, without limitation, abaca, alfalfa, almond, anise seeds, apple, apricot, areca, arracha, arrowroot, artichoke, asparagus, avocado, bajra, bambara groundnut, banana, barley, beans, red beet, sugar beet, bergamot, betel nut, black pepper, black wattle, blackberries, blueberry, brazil nut, breadfruit, broad bean, broccoli, broom millet, broom sorghum, brussels sprouts, buckwheat, cabbage, cacao, cantaloupe, caraway seeds, cardamom, cardoon, carob, carrot, cashew nuts, cassava, castor bean, cauliflower, celeriac, celery, chayote, cherry, chestnut, chickpea, chicory, chili, cinnamon, citron, citronella, clementine, clove, clover, cocoa, coconut, cocoyam, coffee, cola nut, colza, corn, cotton, cottonseed, cowpea, cranberry, cress, cucumber, currants, custard apple, dasheen, dates, drumstick tree, durra, durum wheat, earth pea, edo, eggplant, endive, fennel, fenugreek, fig, filbert, fique, flax, flax seed oil, formio, garlic, geranium, ginger, gooseberry, gourd, gram pea, grape, grapefruit, grass esparto, grass, orchard, grass, sudan, groundnut, guava, guinea corn, hazelnut, hemp, hempseed, henequen, henna, hop, horse bean, horseradish, hybrid maize, indigo, jasmine, jerusalem artichoke, jowar, jute, kale, kapok, kenaf, kohlrabi, lavender, leek, lemon, lemon grass, lentil, lespedeza, lettuce, lime, sour, lime, sweet, linseed, liquorice, litchi, loquat, lupine, macadamia, mace, maguey, maize, mandarin, mangel, mango, manioc, maslin, medlar, melon, broom millet, bajra millet, bulrush millet, finger millet, foxtail millet, Japanese millet, pearl millet, proso millet, mint, mulberry, mushrooms, mustard, nectarine, New Zealand flax, niger seed, nutmeg, oats, okra, olive, onion, green, opium, orange, ornamental plants, palm palmyra, kernel oil, palm oil, palm, sago, papaya, parsnip, pea, peach, peanut, pear, pecan nut, pepper, black, persimmon, pigeon pea, pineapple, pistachio nut, plantain, plum, pomegranate, pomelo, poppy seed, potato, potato, sweet, prune, pumpkin, pyrethum, quebracho, queensland nut, quince, quinine, quinoa, radish, ramie, rapeseed, raspberry, redtop, rhea, rhubarb, rice, rose, rubber, rutabaga, rye, ryegrass seed, safflower, sainfoin, salsify, sapodilla, satsuma, scorzonera, sesame, shea butter, sisal, sorghum, durra sorghum, guinea corn, jowar sorghum, sweet sorghum, soybean, soybean hay, spelt wheat, spinach, squash, strawberry, sugarcane, sunflower, sunhemp, swede, sweet corn, sweet lime, sweet pepper, sweet potato, sweet sorghum, tangerine, tannia, tapioca, taro, tea, teff, timothy, tobacco, tomato, trefoil, triticale, tung tree, tree, turnip, arena, vanilla, vetch for grain, walnut, watermelon, wheat, yam, yerba mate, cottonseed, sunflower seed, safflower seed, crambe, camelina, mustard, rapeseed, collard greens, turnip greens, chard, mustard greens, dandelion greens, switchgrass, miscanthus, arundo donax, energy cane, kelp, sugar cane leaves, and tree leaves.

In some embodiments, a pH switchable reagent can be used to purify a target protein from a crude solution derived from plant material. The target protein can be a naturally occurring plant protein, or can be recombinantly expressed in the plant. Examples of proteins that can be purified from plant material using the materials and methods provided herein include, without limitation, ribulose-1,5-bisphosphate carboxylase oxygenase (rubisco), leghemoglobin, non-symbiotic hemoglobin, ribosomal proteins, cytoplasmic actin, seed storage proteins (e.g., albumin, conalbumin, glutenin, gliadin, glutelin, gluten, hordein, prolamine, phaseolin, secalin, triticeae gluten, and zein), hemoglobin, myoglobin, chlorocruorin, erythrocruorin, neuroglobin, cytoglobin, protoglobin, truncated 2/2 globin, HbN, cyanoglobin, HbO, Glb3, and cytochromes, Hell's gate globin I, bacterial hemoglobins, ciliate myoglobins, flavohemoglobins, ribosomal proteins, actin, hexokinase, lactate dehydrogenase, fructose bisphosphate aldolase, phosphofructokinases, triose phosphate isomerases, phosphoglycerate kinases, phosphoglycerate mutases, enolases, pyruvate kinases, proteases, lipases, amylases, glycoproteins, lectins, mucins, glyceraldehyde-3-phosphate dehydrogenases, pyruvate decarboxylases, actins, translation elongation factors, histones, ribulose-1,5-bisphosphate carboxylase oxygenase activase (rubisco activase), glycinins, conglycinins, globulins, vicilins, gluten, glutenin, prolamin, proteinoplast, extensins, collagens, kafirin, avenin, dehydrins, hydrophilins, late embyogenesis abundant proteins, natively unfolded proteins, oleosins, caloleosins, steroleosins or other oil body proteins, vegetative storage protein A, vegetative storage protein B, moong seed storage 8S globulin, pea globulins, pea albumins, and combinations thereof.

For example, a pH switchable reagent can be used to purify ribulose-1,5-bisphosphate carboxylase oxygenase (rubisco), an enzyme that is involved in the process of carbon fixation and is one of the most abundant proteins in plants. rubisco can be isolated from, for example, alfalfa, carrot tops, corn stover, sugar cane leaves, soybean leaves, switchgrass, miscanthus, energy cane, arundo donax, seaweed, kelp, algae, and mustard greens.

In some embodiments, a pH switchable reagent can be used to purify leghemoglobin, a nitrogen or oxygen carrier that is produced in response to plant roots being infected with nitrogen-fixing bacteria. Leghemoglobin is readily available as an unused by-product of commodity legume crops (e.g., soybean and pea). The leghemoglobin in the roots of these crops in the U.S. exceeds the myoglobin content of all the red meat consumed in the country. Leghemoglobin can be obtained from a variety of plants, including legumes species and their varieties. For example, plants such as soybean, fava bean, lima bean, cowpeas, English peas, yellow peas, lupine, kidney bean, garbanzo beans, peanut, alfalfa, vetch hay, clover, lespedeza and pinto bean contain nitrogen-fixing root nodules in which leghemoglobin has a key role in controlling oxygen concentrations.

In some embodiments, a pH switchable ligand as provided herein can bind rubisco at a pH of about 6, and can release rubisco (or bind to rubisco at a lower affinity) at a pH of about 9. In some embodiments, a pH switchable ligand as provided herein can bind rubisco at a pH of about 9, and can release rubisco (or bind to rubisco at a lower affinity) at a pH of about 6. Further, in some embodiments, a pH switchable ligand as provided herein can bind leghemoglobin at a pH of about 6, and can release leghemoglobin (or bind to leghemoglobin at a lower affinity) at a pH of about 9, while in other embodiments, a pH switchable ligand as provided herein can bind leghemoglobin at a pH of about 9, and can release leghemoglobin (or bind to leghemoglobin at a lower affinity) at a pH of about 6. The binding of a pH switchable reagent to a protein such as rubisco or leghemoglobin can be demonstrated at various pH levels by various methods, including measuring the affinity by Bio-Layer Interferometry (BLI), for example.

Libraries of pH switchable ligands and particular switchable ligands of interest can be produced using, for example, methods known in the art. See, for example, the methods described in PCT/US2013/032675, and in Clark et al. (Nature Chem Biol 5(9):647-654, 2009) which hereby are incorporated by reference in their entirety.

In some embodiments, for example, a library of switchable affinity reagents can be produced using DNA-programmed combinatorial chemistry (DPCC). See, for example, Wrenn et al., J. Am. Chem. Soc. 129(43):13137-13143, 2007; and U.S. Pat. No. 7,479,472, each of which is hereby incorporated by reference in its entirety. DPCC can enable selection of affinity reagents with high and selective affinity for native features of a target molecule from a combinatorial library of small molecules. For example, DPCC can be used to synthesize diverse combinatorial libraries of affinity reagents, where each affinity reagent includes a small molecule compound attached to an encoding polynucleotide sequence that encodes a “program” for synthesis of the small molecule compound from an array of precursors. The term “combinatorial library” can refer to a library of molecules containing a large number (e.g., between about 10³ and 10¹²) of different compounds. The compounds can be characterized by different sequences of subunits or precursors, or by a combination of different sequences of side chains and linkages. In some cases, a population of small molecules can have a common parent structure (e.g., a ring structure, triazine, peptoid, or peptide) and a plurality of different R group substituents or ring-structure modifications, each of which can take a variety of forms. In some embodiments, small molecule compounds can be non-oligomeric, such that they do not consist of sequences of repeating similar subunits. In some embodiments, small molecule compounds can be similar in terms of basic structure and functional groups, but can vary in such aspects as chain length, ring size or number, or patterns of substitution. Small molecule precursors can be, for example, amino acids, peptoids, L-peptides, or D-peptides.

In some embodiments, a switchable affinity reagent can include a small molecule compound and an encoding polynucleotide sequence that contains a program for synthesis of the small molecule compound. The encoding polynucleotide sequence can include one or more homology sequences, and each homology sequence can specify the addition of a specific precursor molecule. Homology sequences can be characterized by (a) binding to their corresponding complementary nucleotide sequences at a specified melting temperature under specified solution conditions, and by (b) not cross-hybridizing efficiently with other sequences at the specified melting temperature and specified solution conditions.

The number of homology sequences in an encoding polynucleotide sequence can be based on the desired length of the small molecule compound. In some cases, for example, the number of homology sequences can be between about one and about 50 (e.g., about one, two, three, four, five, six, seven, eight, nine, ten, 15, 20, 25, 30, 35, 40, 45, 50, 1-5, 2-5, 3-6, 1-10, 10-20, 20-30, 30-40, or 40-50). A homology sequence can contain about one to about 50 nucleotides (e.g., about one, two, three, four, five, six, seven, eight, nine, ten, 11, 12, 13, 14, 15, 16, 17, 18, 19, 20, 25, 30, 35, 40, 45, 50, 1-20, 5-25, 10-30, 15-35, or 20-40 nucleotides). In some embodiments, each homology sequence in an encoding polynucleotide sequence can contain a unique sequence, while in other embodiments, two or more homology sequences in the encoding sequence can contain the same sequence. Further, an encoding polynucleotide sequence can include one or more spacer sequences. The number of spacer sequences can vary according to the number of homology sequences. In some embodiments, for example, each pair of adjacent homology sequences can be separated by a spacer sequence. Spacer sequences can contain between about one and about 50 nucleotides (e.g., about one, two, three, four, five, six, seven, eight, nine, ten, 11, 12, 13, 14, 15, 16, 17, 18, 19, 20, 25, 30, 35, 40, 45, 50, 1-20, 5-25, 10-30, 15-35, or 20-40 nucleotides). Each spacer sequence can have an identical nucleotide sequence, or two or more different spacer sequences can be used.

In some embodiments, an encoding polynucleotide sequence also can include a chemical reaction site, which can be located at the 5′ or 3′ end of the polynucleotide sequence. For example, the 5′ nucleotide can be modified with a commercially available reagent that introduces a phosphate group tethered to a linear spacer that can terminate with, e.g., a primary amine or thiol group. The primary amine provides a chemical reaction site on which a small molecule compound can be synthesized. Many different types of chemical reaction sites (in addition to primary amines) can be introduced at the 5′ terminus of the encoding polynucleotide sequence, including chemical reaction sites such as, without limitation, chemical components capable of forming amide, ester, urea, urethane, carbon-carbonyl, carbon-nitrogen, carbon-carbon, olefin, thioether, or disulfide bonds. In the case of enzymatic synthesis, co-factors can be supplied as required for effective catalysis (e.g., the phosphopantetheinyl group useful for polyketide synthesis).

A split-and-recombine strategy, comprising two or more synthetic steps, can be utilized for synthesis of combinatorial libraries. Traditional split-and-recombine strategies for synthesis of combinatorial libraries is described elsewhere (see, e.g., Chen et al., Meth. Enzymol. 267:211-219, 1996; and Ellman and Gallop, Curr. Opin. Chem. Biol. 2:317-319, 1998). For example, in a combinatorial synthesis consisting of i steps, for which j different chemical coupling reactions are performed at each step, jⁱ compounds will be present in the final library. The traditional split and-recombine strategy is carried out using the following steps; (i) at the beginning of each of the i steps, the pool of solid tags is randomly split into j subsets, (ii) each of the j subsets of solid tags is subjected to a different chemical coupling step, and (iii) after the chemical coupling step, the subsets are recombined into a single pool. This recombined pool is again randomly divided into j subsets (specifically as in (i) above) at the beginning of the next step in the library synthesis. In the synthesis of peptide libraries, for example, the coupling step is the addition of an amino-acid active ester to a free amine group on the solid tag. Each of the j subsets is coupled to a different amino acid (e.g., alanine coupled to subset #1, arginine to subset #2, cysteine to subset #3, etc.). Thus, a split-and-recombine synthesis of 10 synthetic steps, with 10 coupling reactions at each step, would yield a final library size of 10¹⁰.

A compound library can be split into subsets at each step of the split-and-recombine combinatorial synthesis by differential hybridization of an encoding polynucleotide sequence containing one or more homology sequences to complementary sequences bound to a solid support (e.g., polystyrene beads). The complementary sequences to each homology sequence of the encoding polynucleotide sequence can be synthesized. In some embodiments, the 5′ base of each complementary sequence can be modified with a commercially available reagent that introduces a phosphate group tethered to a linear spacer (e.g., a linear spacer having six carbons and terminating with a thiol group). Each of the thiol-bearing complementary sequences can be immobilized, for example, through a thioether linkage to a macroporous resin (e.g., polystyrene) bearing electrophilic bromoacetamide groups. Thus, a number of affinity resins can result, each bearing a unique complementary sequence. Each of the affinity resins can be loaded into its own column. The columns can have luer-lock fittings at either end, and can be connected in a linear sequence.

A first split can be performed by contacting a library of encoding polynucleotide sequences with the above-described affinity resins, for example. In some embodiments, the contacting can include pumping a high-salt aqueous solution containing the entire library of encoding polynucleotide sequences cyclically over the linear sequence of affinity columns under high stringency conditions (see, e.g., Southern et al., Nucl. Acids Res. 22(8):1368-1373, 1994), using a peristaltic pump and for a time sufficient for all of the encoding polynucleotide sequences to hybridize to the complementary sequences bound to the columns. The split can be completed by breaking the luer-lock linkages between the affinity columns. At this point, the different encoding polynucleotide sequences have been divided into physically separate subsets on the basis of homology sequences contained with the encoding polynucleotide sequences.

Each subset of encoding polynucleotide sequences formed by hybridization as described above can then be subjected to a different synthetic coupling reaction. The methods employed in the synthetic coupling reaction can vary according to the desired small molecule compound. For example, an amino terminal blocking group (e.g., a fluorenylmethoxycarbonyl (FMOC) group), which can be added and removed, can be used in the synthesis of a small molecule compound containing peptide subunits.

An exemplary procedure for synthesizing small molecule compounds having peptide or amino acid subunits or precursors is as follows. Encoding polynucleotide sequences bound to affinity columns can be eluted from the affinity columns with 10 mM NaOH and 0.005% Triton X-100. The polynucleotide sequences can be transferred onto chemistry columns (e.g., hydroxyapatite resin columns with binding in 300 mM CaCl2, or DEAE Sepharose fastflow columns with binding in 10 mM acetate at pH 5.0 with 0.005% triton). The encoding polynucleotide sequences can remain non-covalently bound to the hydroxyapatite or sepharose resin in numerous organic solvents (e.g., DMF, acetonitrile, ethanol, and mixtures of such solvents with water). Thus, organic reagents can be flowed over the columns and reacted with sites on the encoding nucleotide sequences in the same manner that conventional solid phase chemical synthesis is carried out. Accordingly, a different Fmoc-protected amino-acid preactivated with N[(1H-benzotriazol-1-yl) (dimethylamino) methylene]-N-methylmethanaminium tetrafluoroborate (TBTU) or as an N-hydroxy succinimide ester in DMF can be flowed over each hydroxyapatite or sepharose column, resulting in acylation of the primary amines of the chemical reaction site on each of the hydroxyapatite or sepharose columns with an Fmoc-protected amino acid (see, Albericio and Carpino, Meth. Enzymol. 289:104-126, 1997). Following acylation, the Fmoc group can removed from the newly added amino acid by flowing a piperidine/DMF solution over the hydroxyapatite or sepharose columns, thus presenting a new primary amine ready for the next coupling step.

An exemplary procedure for synthesizing small molecule compounds containing peptoid subunits or precursors is as follows. The encoding polynucleotide sequences bound to the affinity columns can be transferred onto chemistry columns (e.g., DEAE-sepharose columns), and washed with DEAE bind buffer (10 mM acetic acid, 0.005% Triton-X100), water, methanol, or a combination thereof. The chemistry columns then can be incubated one or more times with 150 mM DMT-MM (4-(4,6-dimethoxy-1,3,5-triazin-2-yl)-4-methylmorpholinium chloride) and 100 mM sodium chloroacetate in distilled methanol for about 20 minutes. The columns then can be washed with methanol and incubated with a solution containing a desired peptoid subunit. The column can be microwaved for about 20 seconds one or more (e.g., one to six) times during the peptoid incubation. The column then can be washed, for example, with DMSO, DEAE bind buffer, or a combination thereof.

An entire affinity reagent library can be synthesized by carrying out alternate rounds of library splitting and chemical and/or biochemical coupling to each subset of encoding nucleotide sequences.

Methods for generating affinity reagent libraries can be modified in order to generate switchable affinity reagents, where the switchable affinity reagents include a small molecule compound and an encoding polynucleotide sequence. The small molecule compound can be synthesized from subunit or precursors as described herein, for example. In some embodiments, the precursors can be chosen to bias the synthesis toward a family of molecules with a pKa of about 7, so they can be neutrally charged at about pH 9 and positively charged at about pH 5. The affinity reagents can be incubated with a crude mixture of non-specific proteins (e.g., cell lysates from Escherichia coli), which can eliminate molecules with non-specific affinity for proteins. The remaining library can then be incubated at a binding pH as described herein (e.g., a pH of about 9) with a particular target protein attached to a solid support. Affinity reagents that fail to bind to the target protein can be washed away. The buffer pH then can be adjusted to a release pH as described herein (e.g., a pH of about 5), and the bound affinity reagents that elute can be collected. It should be understood that the stated pH values are provided merely as examples and that a variety of binding and release pH combinations are contemplated.

This selection can be reiterated to obtain progressively greater enrichment of the affinity reagents that perform best in the selection (e.g., binding at about pH 9 and eluting at about pH 5). The selected pool of affinity reagents can be identified by sequencing the attached DNA molecules. The affinity reagents then can be resynthesized and further characterized for their specificity to the target protein and performance under actual purification conditions. Any number of iterations can be employed to select the affinity reagents (e.g., one, two, three, four, five, six, seven, eight, nine, ten, 11, 12, 13, 14, 15, 16, 17, 18, 19, 20 or more than 20 iterations can be performed).

Using the methods described above, pH switchable reagents specific for particular targets (e.g., rubisco and leghemoglobin) have been identified. In some embodiments, therefore, a pH switchable ligand can have a structure as depicted in Formula (I):

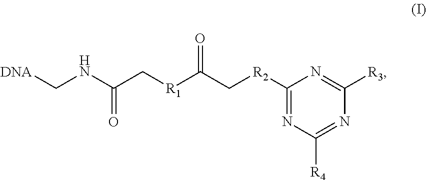

where the R1-R4 groups of Formula (I) correspond, respectively, to the R1-R4 groups listed in Tables 1-5, with the attachment point between the R groups in the tables and the structure of Formula I being through the N of the R groups in the tables.

In some cases, a pH switchable ligand can have a structure as depicted in Formula (II):

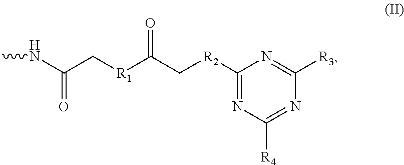

where the R1-R4 groups of Formula (II) correspond, respectively, to the R1-R4 groups listed in Tables 1-5, with the attachment point between the R groups in the tables and the structure of Formula II being through the N of the R groups in the tables. The wavy line indicates the point at which the pH switchable ligand can be attached to a substrate (e.g., a resin or a bead).

In some embodiments, the structure of the backbone of a pH switchable ligand can be depicted as shown in Formula (III):

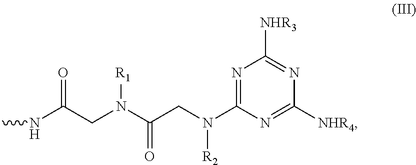

where the N—R1 to N—R4 groups of Formula (III) correspond, respectively, to the R1-R4 groups listed in Tables 1-5, such that the N of each N—R group in Formula (III) is the N of an R group in the tables. The wavy line indicates the point at which the pH switchable ligand can be attached to a substrate (e.g., a resin or a bead).

Thus, in some embodiments, a pH switchable ligand can include R groups as listed in Table 1.

TABLE 1

| | R1 | R2 | R3 | R4 |
|---|---|---|---|---|
| 16 | 3-fluoro-(2-phenyl | 2-amino-N- | 2-methyl | (4-bromophenyl) |
| | ethanamine) | cyclopropylacetamide | butylamine | methanamine |
| 1 | 2-methoxy- | 2-(3-fluorophenyl) | 4-aminobutan-1- | 2-amino-1- |
| | ethylamine | ethanamine | ol | phenylethanol |

In some embodiments, a pH switchable ligand as provided herein can bind rubisco at a pH of about 6, and can release rubisco (or bind to rubisco at a lower affinity) at a pH of about 9. Table 2 lists examples of R groups for pH switchable ligands that can bind rubisco at pH about 6 and release at pH about 9.

TABLE 2
R groups for pH switchable ligands that can bind rubisco at pH 6 and release at pH 9

| | R1 | R2 | R3 | R4 |
|---|---|---|---|---|
| 1501 | (R)-1-(3-methoxy- | (S)-(−)-1- | butylamine | 2-aminoethyl |
| | phenyl) ethylamine | phenylethylamine | | isopropyl ether |
| 1502 | 3-(methylthio) | 4-bromobenzylamine | (S)-(−)-1-(3-methoxy- | butanoic acid, 2- |
| | propylamine | | phenyl) ethylamine | amino-3-hydroxy- |
| 1503 | (2,2-dimethyl-[1,3]- | 4-(difluoromethoxy) | 3-fluoro-tyrosine | 2-methylbutylamine |
| | dioxolan-4-yl)- | benzylamine | | |
| | methylamine | | | |
| 1504 | 3-(methylthio) | 4-pyridinemethanamine, | 2-amino-1- | ethanone, 2-amino-1- |
| | propylamine | 2-(dimethylamino)- | methoxybutane | (4-morpholinyl)- |
| 1505 | 4-(2-aminoethyl) | 4-pyridinemethanamine, | 4-aminotetrahydro | isopropylamine |
| | morpholine | 2-(dimethylamino)- | pyran | |
| 1506 | benzylamine | (R)-(−)-2-amino-1- | 2-amino-3-methyl-3- | 3-chloro-2,6- |
| | | butanol | sulfanylbutanoic acid | difluorobenzylamine |
| 1507 | ethanone, 2-amino-1- | aminomethane sulfonic | 3 -phenylpropylamine | 2-amino-1- |
| | (4-morpholinyl)- | acid | | methoxybutane |
| 1508 | 3-amino-1-propanol | piperazine | 4-aminopiperidine | 2-amino-1- |
| | | | | methoxybutane |
| 1509 | 3-fluoro- | acetamide, 2-amino-n- | 4-bromobenzylamine | 2-methylbutylamine |
| | phenethylamine | cyclopropyl- | | |
| 1510 | 1-(3-aminopropyl)- | 2,5-dimethylaniline | 4-ethoxyaniline | 2-(1-cyclohexenyl)- |
| | pyrrolidine | | | ethylamine |
| 1511 | 3-aminopropionitrile | 2-ethoxyaniline | 5-amino-1-pentanol | 4-amino-1-butanol |
| 1512 | 2-(3,4-dimethoxy- | 4-pyridinemethanamine, | 1-(3-aminopropyl)-2- | 2-chlorobenzylamine |
| | phenyl) ethylamine | 2-(dimethylamino)- | pipecoline | |

In some embodiments, a pH switchable ligand as provided herein can bind rubisco at a pH of about 9, and can release rubisco (or bind to rubisco at a lower affinity) at a pH of about 6. Table 3 lists examples of R groups for pH switchable ligands that can bind rubisco at pH 9 and release at pH 6.

TABLE 3
R groups for pH switchable ligands that can bind rubisco at pH 9 and release at pH 6

| | R1 | R2 | R3 | R4 |
|---|---|---|---|---|
| 2101 | 2-(3,4-dimethoxy- | N-tert-butoxycarbonyl- | tetrahydro- | 3-amino-1-propanol |
| | phenyl)ethylamine | 3-aminopropanamine | furfurylamine | |
| 2102 | 2-(2-aminoethoxy)- | 3-chloroaniline | 9-aminofluorene | butanoic acid, 2- |
| | ethanol | | | amino-3-hydroxy- |
| 2103 | acetamide, 2-amino-N- | 4-methoxy- | pentanoic acid, 5- | (R)-(+)-1- |
| | cyclopropyl- | benzylamine | amino- | phenylethylamine |
| 2104 | (2,2-dimethyl-[1,3]- | 2-chlorobenzylamine | 3-phenylpropylamine | butanoic acid, 2- |
| | dioxolan-4-yl)- | | | amino-3-hydroxy- |
| | methylamine | | | |
| 2105 | 4-methoxybenzylamine | 2-(4-chlorophenyl)- | benzeneacetic acid, | 3-(2-aminoethyl)- |
| | | ethylamine | α-amino-4-fluoro- | pyridine |
| 2106 | 2-phenoxyethylamine | 1-leucine-4-nitroanilide | 2-(2- | N-acetylethylene- |
| | | | furylmethylthio)- | diamine |
| | | | ethanamine | |
| 2107 | benzylamine | 2-chlorobenzylamine | 2-amino-1- | 2-(2-aminoethoxy)- |
| | | | methoxybutane | ethanol |
| 2108 | N-(3′-aminopropyl)-2- | N,N-diethyl-1,3- | 3-chlorobenzylamine | (2S)-4-amino-2- |
| | pyrrolidinone | propanediamine | | hydroxy-butyric acid |
| 2109 | 2-(aminomethyl)pyridine | 2,6-difluoroaniline | cyclopentylamine | butanoic acid, 2- |
| | | | | amino- |
| 2110 | 2-(4-chlorophenyl)- | S-(2-aminoethyl)- | 2-chlorobenzylamine | 2-(2-aminoethoxy)- |
| | ethylamine | isothiouronium | | ethanol |
| | | bromide | | |
| 2111 | propargylamine | 3-amino-1-propanol | furfurylamine | 2-amino-1- |
| | | | | methoxybutane |
| 2112 | 3-fluorobenzylamine | 2-(1H-indol-3-yl)- | 4-bromobenzylamine | furfurylamine |
| | | ethanamine | | |
| | | (tryptamine) | | |

In some embodiments, a pH switchable ligand as provided herein can bind leghemoglobin (e.g., leghemoglobin from Glycine max) at a pH of about 6, and can release leghemoglobin (or bind to leghemoglobin at a lower affinity) at a pH of about 9. Table 4 lists examples of R groups for pH switchable ligands that can bind leghemoglobin at pH about 6 and release leghemoglobin at pH about 9.

TABLE 4
R groups for pH switchable ligands that can bind leghemoglobin at pH 6 and release at pH 9

| | R1 | R2 | R3 | R4 |
|---|---|---|---|---|
| 1801 | N-(3′-aminopropyl)-2- | alpha-phenyl- | N-(3-aminopropyl)- | 1,3-dimethylbutylamine |
| | pyrrolidinone | glycinonitrile | morpholine | |
| 1802 | 2-phenylpropylamine | 2-amino-1- | 2-amino-3,3- | benzoic acid, 2- |
| | | methoxybutane | dimethylbutane | (aminomethyl)- |
| 1803 | N-tert- | 3-N-propoxy- | 2-fluoroethanamine | (S)-(−)-beta-methyl- |
| | butoxycarbonyl-3- | propylamine | | phenethylamine |
| | aminopropanamine | | | |
| 1804 | 2-(aminomethyl)-1- | N,N,2,2-tetramethyl- | 5-amino-1-pentanol | 2-(aminomethyl) pyridine |
| | ethylpyrrolidine | 1,3-propanediamine | | |
| 1805 | 3-amino-1-propanol | 3-isopropoxy- | 8-quinoline- | N-acetylethylene-diamine |
| | | propylamine | methanamine | |
| 1806 | acetamide, 2-amino- | isopropylamine | N,N-dimethyl-1,3- | N-(3′-aminopropyl)-2- |
| | N-cyclopropyl- | | propanediamine | pyrrolidinone |
| 1807 | 2-thiophene- | 2-fluoroethanamine | glutamic acid | 4-bromobenzylamine |
| | methylamine | | | |
| 1808 | 1-amino-3,3- | 4-aminopyridine | 3-(aminomethyl)- | N,N,2,2-tetramethyl-1,3- |
| | diethoxypropane | | pyridine | propanediamine |
| 1809 | butylamine | 2-(1-cyclohexenyl)- | acetamide, 2-amino- | 2-aminoethyl isopropyl |
| | | ethylamine | N-cyclopropyl- | ether |
| 1810 | 4-(2-aminoethyl)- | 3-isopropylaniline | 1-(2-aminoethyl)- | 4-(aminomethyl)-pyridine |
| | pyridine | | imidazolidin-2-one | |
| 1811 | N-(3′-aminopropyl)-2- | 3-chlorobenzylamine | 3-(methylthio)- | sec-butylamine |
| | pyrrolidinone | | propylamine | |
| 1812 | 2-methylbutylamine | furfurylamine | 3-fluoro-5- | 2-aminoethyl isopropyl |
| | | | (trifluoro-methyl) | ether |
| | | | benzylamine | |

In some embodiments, a pH switchable ligand as provided herein can bind leghemoglobin (e.g., leghemoglobin from Glycine max) at a pH of about 9, and can release leghemoglobin (or bind to leghemoglobin at a lower affinity) at a pH of about 6. Table 5 lists examples of R groups for pH switchable ligands that can bind leghemoglobin at pH about 9 and release leghemoglobin at pH about 6.

TABLE 5
R groups for pH switchable ligands that can bind leghemoglobin at pH 9 and release at pH 6.

| | R1 | R2 | R3 | R4 |
|---|---|---|---|---|
| 1901 | 2-chloro-benzylamine | 4-aminobenzamide | 4-(2-aminoethyl)-1- | 3-(trifluoromethyl)- |
| | | | benzylpiperidine | benzylamine |
| 1902 | N,N,2,2-tetramethyl- | 2,5-dimethylaniline | 3-methoxytyramine | 3-aminopyrrolidine |
| | 1,3-propanediamine | | | |
| 1903 | 2-(1H-indol-3-yl)- | 3-chlorobenzylamine | 1-amino-2-butanol | 1,3-dimethylbutylamine |
| | ethanamine | | | |
| | (tryptamine) | | | |
| 1904 | 4-(2-aminoethyl)-1- | 4-iodobenzylamine | D-Phenylalanine, 3- | 4-aminotetra-hydropyran |
| | benzylpiperidine | | fluoro- | |
| 1905 | 2-(4-chlorophenyl)- | 3-bromo-4-methoxy- | (S)-2-amino-1- | (S)-(−)-beta-methyl- |
| | ethylamine | phenethylamine | phenylethanol | phenethylamine |
| 1906 | 3-fluorobenzylamine | (R)-(−)-1-aminoindan | 3-N-propoxy- | (2,2-dimethyl-[1,3]- |
| | | | propylamine | dioxolan-4-yl)- |
| | | | | methylamine |
| 1907 | 4-fluoro- | 2-phenylpropylamine | N-(3-aminopropyl) | butanoic acid, 2-amino- |
| | phenethylamine | | morpholine | |
| 1908 | 3-furanmethanamine | 4-iodobenzylamine | benzylamine | ethanone, 2-amino-1-(4- |
| | | | | morpholinyl)- |
| 1909 | 5-aminomethyl-2- | 3-aminopyridine | asparagine | 3-(2-aminoethyl)- |
| | chloropyridine | | | pyridine |
| 1910 | isobutylamine | 5-aminomethyl-2- | 3-n-propoxy- | 4-bromobenzylamine |
| | | chloropyridine | propylamine | |
| 1911 | 3-fluoro- | 4-aminopyridine | meta- | benzylamine |
| | phenethylamine | | aminobenzoylamine | |
| 1912 | 3-fluorobenzylamine | 4-bromobenzylamine | beta alanine | chloro |
| | | | napthylamide HBr | |

Further examples of pH switchable ligands are described in, for example, PCT/US2013/032675, which published as WO 2013/138793 and is hereby incorporated by reference in its entirety.

In some embodiments, a pH switchable ligand can be coupled to a solid or semi-solid support (e.g., a resin) for use in purification. Table 6 lists examples of specific coupling reagents that can be used to couple ligands as described herein (e.g., pH switchable ligands) to solid supports, while Table 7 lists examples of resins to which the ligands coupled. Useful resins can be suitable for chromatography applications, for example.

In some embodiments, a ligand can be coupled to a resin through a linker group, which can increase the availability of the ligand to the target. Table 8 lists examples of specific linkers that can be coupled to the ligands (e.g., pH switchable ligands) provided herein.

In some embodiments, a switchable affinity ligand can be synthesized directly on a resin. For example, cyanogen bromide activated sepharose (CNBr sepharose) can be reacted with an excess amount of a long chain diamine as a linker. The resulting amine-terminated resin can be reacted in sequence with a methanolic solution of sodium chloroacetate and an amide coupling reagent (4-(4,6-dimethoxy-1,3,5-triazin-2-yl)-4-methylmorpholinium chloride, abbreviated as DMTMM) to endcap the amine with a chloroacetamide, followed by treatment with a primary amine, and then repetition of the first step to endcap the amine with a chloroacetyl group. The product then can be treated with a (possibly) different primary amine.

The resulting alkylaminemethylacetamido compound can be treated with cyanuric chloride at 0° C., followed by treatment by with a primary amine to displace a chloro atom on the triazine in a SNAr reaction at 20° C., followed by treatment with a final amine to displace the last remaining chloride in the triazine. The resin can be washed liberally with methanol, followed by 0.1 M sodium carbonate (pH 9) and stored in 20% ethanol/water.

In some embodiments, a switchable affinity ligand (e.g., a pH switchable ligand) can be synthesized in solution. Such synthesis can include triazine synthesis of ligands. In solution synthesis can be used to make gram or larger quantities of a pH switchable ligand. Ligands synthesized using solution synthesis methods can be coupled to a solid substrate (e.g., a resin).

TABLE 6
Examples of coupling reagents

4-(4,6-Dimethoxy-1,3,5-triazin-2-yl)-4-methylmorpholinium chloride
N-(3-Dimethylaminopropyl)-N′-ethylcarbodiimide hydrochloride
Dicyclohexylcarbodiimide
2-chlorodimethoxytriazine

TABLE 7
Resins

merrifield resin
CNBr sepharose
Sepharose 4B
CM Sepharose
polypropylene graft maleic anhydride
poly alt-methylvinylethermaleic anhydride
Poly(styrene-co-maleic anhydride), cumene terminated
Polystyrene-co-maleic anhydride
polyacrylic acid (450 kD)
polyacrylic acid (3000 kD)
polyacrylic acid in water (100 kD)
hydroxyapatite, rgt grade powder
calcium phosphate, purum
tribasic calcium phosphate
carboxymethylcellulose (Chisso Cellufine Amino)
Carboxymethylcellulose
Itochu WK100
Itochu PK216

TABLE 8
Linker groups

trimethylolethane
trimethylolpropane
pentaerythritol
hexamethylenediamine
tetramethylenediamine
trimethylenediamine
ethylenediamine
p-phenylenediamine
m-phenylenediamine
isophorone diisocyanate
hexamethylenediisocyanate
toluene 2,4-diisocyanate

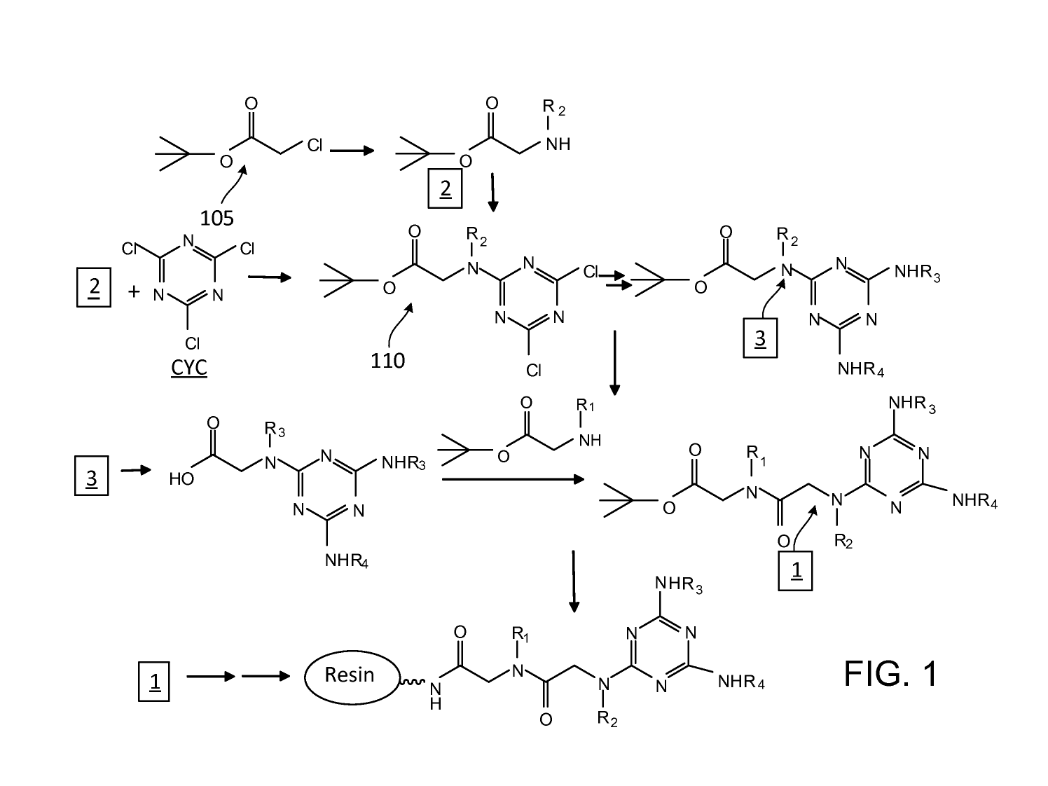

FIG. 1 depicts an example of a solution synthesis according to the methods described herein. The synthesis starts with reaction of chloroacetate ester 105 with an amine to make a substituted glycinate 2. This reaction can be general for a variety of amines. The glycinate can be treated with cyanuric chloride (CYC) to make a monosubstituted triazine 110. Subsequent chlorides can be displaced successively to form trisubstituted triazine 3. Ester hydrolysis to generate an acid, followed by amide coupling with a separate glycinate can form an extended derivative, 1. This derivative may be coupled as the acid to any appropriate resin or solid support to be used for the purpose of protein purification.

This document also provides methods for purifying target molecules (e.g., proteins or other compounds) using one or more switchable affinity reagents. The methods can include (a) applying a composition containing a target molecule to a substrate having a switchable affinity reagent at a binding pH, such that the target molecule binds to the switchable affinity reagent; and (b) applying a solution at a release pH to the substrate, such that the target molecule is released from (or binds with lower affinity to) the switchable affinity reagent. In some embodiments, the methods also can include a washing step in which a wash solution is applied to the substrate bound to the target protein, where the wash solution is at or near the binding pH. The wash step can be used to remove one or more undesirable components (e.g., undesirable proteins or contaminants).

In some embodiments, a column purification can be performed in which a crude solution containing a target molecule is run through a column containing beads or a resin to which one or more switchable affinity reagents are linked. The crude solution can be at a binding pH, at which the target will bind to the switchable affinity reagent with relatively high affinity, and at which other components of the crude solution will pass through the column and can be collected and/or disposed. The crude solution can be run over the column one or more times. It is noted that running a crude solution over a column two or more times can increase the amount of target molecule that is purified from the crude solution.

In some embodiments, a batch purification can be performed in which beads linked to one or more switchable affinity reagents can be added to a crude solution containing a target molecule. The crude solution can be at a binding pH, at which the target will bind to the switchable affinity reagent with relatively high affinity. Following an incubation period with optional mixing, the beads can be collected and removed from the crude solution, along with any target proteins bound to the switchable affinity reagent(s). As for column purification, batch purification methods can include one or more wash steps performed with a solution that is at or near the binding pH.

In both column and batch purifications, the one or more target molecules can be released from the switchable affinity reagent(s) using a solution at a release pH, at which the target will not bind to the switchable affinity reagent, or will bind to the switchable affinity reagent with relatively low affinity. Further, both column and batch purification methods can be used to produce pure, concentrated solutions of the target molecules.

Purification of target proteins (e.g., plant-derived target proteins) using pH switchable reagents can be designed for use on a massively large scale. For example, isolation of high fructose corn syrup, antibiotics, or industrial proteins can occur in columns greater than 4 meters in diameter. Such columns often are used for process development and manufacturing. Columns used for purifying plant target proteins using pH switchable reagents as described herein can be up to about one, two, three, four, five, six, seven, eight, nine, ten, or more than ten times the size of the current largest columns, and the amount of target protein recovered can range from at least 0.1 g to at least 100000 grams or more (e.g., at least 0.1, 0.5, 1, 5, 10, 20, 50, 100, 200, 500, 1000, 5000, 10000, 50000, 100000, or more than 100000 grams). The concentration of recovered target protein can be at least 0.1 mg/mL (e.g., 0.5 mg/mL, 1 mg/mL, 5 mg/mL, 10 mg/mL, 20 mg/mL, 50 mg/mL, 100 mg/mL, 200 mg/mL, or 500 mg/mL).

The purity of a target sample can be assessed using standard protein purity detection techniques, such as SDS page, mass spectrometry, spectroscopy, or other molecular biology techniques that are known to those of skill in the art.

In some instances, a pH switchable reagent may leach off a column and be carried into the target protein sample that will be used in downstream processes to produce a composition or consumable food product. This suggests that the consumable food product may contain minute quantities of the pH switchable reagent. In some embodiments, the amount of pH switchable reagent and/or pH switchable reagent resin in a composition or consumable product is at most 5000 ppm target protein (e.g., at most 1, 100, 500, 1000, 2000, or 5000 ppm target protein), or at most 10 percent (e.g., at most 10⁻⁹, 10⁻⁸, 10⁻⁷, 10⁻⁶, 10⁻⁵, 10⁻⁴, 10⁻³, 0.01, 0.05, 0.1, 0.5, 1, 5, or 10 percent) weight/volume of the purified target protein composition. In some embodiments, the amount of leached pH switchable reagent and/or pH switchable reagent resin is at most 1000 parts per billing (ppb) (e.g., at most 0.001, 0.005, 0.01, 0.05, 0.1, 0.5, 1, 5, 10, 50, 100, 500, or 1000 ppb). Further, in some embodiments, the purified target protein represents more than 10 percent (e.g., 20, 30, 40, 50, 60, 70, 80, 85, 90, 91, 92, 93, 94, 95, 96, 97, 98, 98.5, 99, 99.5, 99.9, or 99.95 percent) of the weight/volume of the purified target protein composition. In some embodiments, the purified target protein is increased by a factor of two or more (e.g., three or more, five or more, ten or more, 20 or more, 50 or more, 100 or more, or 1000 or more) relative to the source material from which the specified protein was isolated.

A purified target protein can be substantially free of celluloses (e.g., lignin, hemicellulose, and cellulose) and complex polysaccharides (e.g., arabinose, xylose, arabinoxylans, cellulose, lignin, hemicellulose, chitin, pectin, amylose, or amylopectins). Cellulose is the polysaccharide responsible for the structural integrity of the plant cell wall, and may not be desirable in a consumable product because they are only partially digestible by humans. Complex polysaccharides also can be found in the plant cell walls, and may not be desirable in a consumable product.

A pure target protein can be substantially free of colorants and/or odorants, such that it contains at most only trace amounts of colorants and/or odorants. Colorants include, for example, anions, cations, salts, metals, alkali metals, metalloids, and transition elements. Colorants can be of a type related to chlorophylls, such as chlorins, chlorophyll a, chlorophyll b, chlorophyll c1, chlorophyll c2, chlorophyll d, and chlorophyll f. In some embodiments, the amount of colorant and/or odorant in a purified target protein preparation will be at most 100000 ppm target protein (e.g., at most 1, 100, 500, 1000, 2000, 5000, 10000, or 100000 ppm target protein), or at most 10 percent (e.g., at most 0.05, 0.1, 0.5, 1, 2, 3, 4, 5, 6, 7, 8, 9, or 10 percent) weight/volume of the purified target protein composition. In some embodiments, the purified target protein will represent more than 90 percent (e.g., 91, 92, 93, 94, 95, 96, 97, 98, 98.5, 99, 99.5, 99.9, or 99.95 percent) of the weight/volume of the purified target protein preparation.

This document also provides methods for removing one or more undesirable components (e.g., contaminants or toxins) from a mixture using one or more switchable affinity reagents, wherein the one or more switchable affinity reagents bind specifically to the undesirable components under a first set of conditions (e.g., a binding pH), and do not bind or bind with a lower affinity under a second set of conditions (e.g., a release pH). The switchable affinity reagents can be bound to a solid support or substrate such as a bead or a resin, which can be used in column or batch purifications. In some embodiments, a mixture containing one or more undesirable components can be applied to a column, where the column is packed with beads or resin linked to one or more switchable affinity reagents, and where the one or more switchable affinity reagents specifically bind to the one or more undesirable components under a first set of conditions (e.g., a binding pH), and do not bind (or bind less strongly) under a second set of conditions (e.g., a release pH). Prior to running the mixture over the column, the column and/or the mixture can be adjusted to the first set of conditions (e.g., the binding pH), such that the mixture that passes through the column will have all, substantially all, or a portion of the undesired components removed. The mixture can be passed through the column one or more additional times in order to further deplete the mixture of the undesirable components. A second solution under the second set of conditions (e.g., the release pH) can be passed over the column to regenerate the beads or resin by releasing the undesirable components from the switchable affinity reagents. Such methods can be useful, for example, for removing toxins from an environment, or for removing allergens or other undesirable components from a mixture.

The invention will be further described in the following examples, which do not limit the scope of the invention described in the claims.

### EXAMPLES

### Example 1

### Synthesis of pH Switchable Ligand 16 (Listed in Table 1)

1. Attachment of Linker to CNBr Sepharose.

In a 500 mL round bottomed flask, 5 grams of wet (approximately 50% water) of CNBr Sepharose was treated with 4.4 g of 4,7,10-trioxa-1,13-tridecanediamine in 44 mL water. The flask was attached to a Roto-Vap and rotated for 3 hours. At the end of this time, the solids were filtered and washed with water, followed by a wash with pH 9 sodium carbonate solution (0.1 M). At this point, the resin preparation was ready for immediate use. Storage for longer than 2 days was done in 20% ethanol/water at 4° C.

2. Chloroacetylation of Amino Endcapped CNBr Sepharose Resin.

To a BioRad minicolumn containing about 1 mL of wet resin prepared as in Step 1, 6 ml methanol was added, after which the solvent was allowed to drain. The column was capped so flow was stopped. Separately, 6.0 mL of a 0.2 M solution of sodium chloroacetate and 3.7 mL of a 0.3 M solution of DMTMM (4-(4,6-Dimethoxy-1,3,5-triazin-2-yl)-4-methylmorpholinium chloride) were mixed together to form a solution. This solution was added to the CNBr Sepharose, and mixed thoroughly in the plastic column, and allowed to stand at ambient temperature for at least 15 minutes. At the end of that time, the cap was removed from the bottom of the column and the contents were allowed to drain. Methanol (6 mL) was added to the top of the column and allowed to drain through.

3. Amination of Chloroacetylated Resin Followed by a Second Chloroacetylation.

The column was capped and a 2 M solution of 3-fluorophenylethylamine in DMSO (0.53 mL) was added to the top of the column and mixed thoroughly. This was allowed to stand a total of 2 hours with a mixing step half way between. The cap was removed and the DMSO was allowed to drain. Methanol (6 mL) was added to the top of the column to wash out amine and DMSO. This was followed by 0.1 M aqueous sodium carbonate (pH 9). After this step, Step 2 (above) was repeated starting with the methanol wash.

4. Second Amination of Resin.

To the methanol wet resin from Step 3 above was added a 1M solution of 2-amino-N-cyclopropyl-acetamide in DMSO. The procedure was otherwise similar to Step 3 with a stop after the aqueous sodium carbonate wash.

5. Triazinylation.

An 8.3% cyanuric chloride solution was made by suspending cyanuric chloride in acetonitrile, followed by filtration through a syringe filter (0.45 um). Separately, the resin was washed with 6 mL acetonitrile and drained. The column was capped to prevent drainage and 3.1 ml of cyanuric chloride solution was added to the top of the resin followed by 0.192 ml diisopropylethylamine. The mixture was mixed thoroughly with the resin and allowed to stand for an hour in a 4° C. cold room. The cap was then removed, the column was allowed to drain, and the column was washed with 12 mL acetonitrile.

6. Second Triazine Addition.

To the resin in the capped column from Step 5 was added a mixture of 0.028 mL 2-methylbutylamine and 1.1 mL of 1M diisopropylethylamine in acetonitrile. The solid resin and solution were mixed thoroughly and allowed to stand at ambient temperature for an hour. The cap was then removed from the column and the solvent was drained, and the column was washed with 6 mL acetonitrile.

7. Third Triazine Addition.

To the resin in the capped column from Step 6 was added 6 mL acetonitrile to resuspend the solid. The contents were poured into a scintillation vial and allowed to settle. The acetonitrile on top was removed and a mixture of 56 mg mL 4-bromobenzylammonium hydrochloride, and 1.1 mL of 1M diisopropylethylamine in acetonitrile was added. The vial was capped and the whole mixture was swirled gently and placed in a 60° C. oven for a total of three hours. The mixture was swirled on an hourly basis. At the end of this time, the vial was removed from the oven and allowed to cool before it was opened. The contents were poured into a minicolumn and drained. Fresh acetonitrile (6 mL) was added to the vial to wash it out and this was added to the solid in the column. The solid was washed with 12 ml 0.1 M sodium carbonate (pH 9), followed by storage in 20% ethanol/water.

### Example 2

### Synthesis of pH Switchable Ligand 1 (Listed in Table 1)

1. and 2. For the First Two Steps See the Preparation of Candidate 16 Above.

3. Amination of Chloroacetylated Resin Followed by a Second Chloroacetylation.

The column was capped and a 2 M solution of 2-methoxyethylamine in DMSO (0.53 mL) was added to the top of the column and mixed thoroughly. This was allowed to stand a total of 2 hours with a mixing step half way between. The cap was removed and the DMSO was allowed to drain. Methanol (6 mL) was added to the top of the column to wash out amine and DMSO. This was followed by 0.1 M aqueous sodium carbonate (pH 9). After this step, Step 2 (see Step 2, Candidate 16 above) was repeated starting with the methanol wash.

4. Second Amination of Resin.

To the methanol wet resin from Step 3 above was added a 1M solution of 3-fluorophenylethylamine in DMSO. The procedure was otherwise similar to Step 3 with a stop after the aqueous sodium carbonate wash.

5. Triazinylation.

An 8.3% cyanuric chloride solution was made by suspending cyanuric chloride in acetonitrile, followed by filtration through a syringe filter (0.45 um). Separately, the resin was washed with 6 mL acetonitrile and drained. The column was capped to prevent drainage and 3.1 ml of cyanuric chloride solution was added to the top of the resin followed by 0.192 ml diisopropylethylamine. The mixture was mixed thoroughly with the resin and allowed to stand for an hour in a 4° C. cold room. The cap was then removed, the column was allowed to drain, and the column was washed with 12 mL acetonitrile.

6. Second Triazine Addition.

To the resin in the capped column from Step 5 was added a mixture of 0.20 mL 4-aminobutanol and 1.1 mL of 1M diisopropylethylamine in acetonitrile. The solid resin and solution were mixed thoroughly and allowed to stand at ambient temperature for an hour. The cap was then removed from the column and the solvent was drained, and the column was washed with 6 mL acetonitrile.

7. Third Triazine Addition.

To the resin in the capped column from Step 6 was added 6 mL acetonitrile to resuspend the solid. The contents were poured into a scintillation vial and allowed to settle. The acetonitrile on top was removed and a mixture of 301 mg 2-amino-1-phenyethanol and 1.1 mL of 1M diisopropylethylamine in acetonitrile was added. The vial was capped and the whole mixture was swirled gently and placed in a 60° C. oven for a total of three hours. The mixture was swirled on an hourly basis. At the end of this time, the vial was removed from the oven and allowed to cool before it was opened. The contents were poured into a minicolumn and drained. Fresh acetonitrile (6 mL) was added to the vial to wash it out and this was added to the solid in the column. The solid was washed with 12 ml 0.1 M sodium carbonate (pH 9), followed by storage in 20% ethanol/water.

### Example 3

### Evidence for pH-Dependent Affinity to Rubisco of Candidates 16 and 1

pH switchable ligands were tested for binding to rubisco by surface plasmon resonance (SPR). The pH switchable ligands (“Candidates”) were synthesized on a 5′-amino-modified, 20-base ssDNA oligo. A complementary 20-base ssDNA oligo with a 5′-biotin modification was purchased from a commercial vendor. First, the 5′-biotin strand of DNA was flowed over a Proteon SPR Streptavidin-coated sensor chip for 60 seconds. The biotin bound the streptavidin, thereby immobilizing the ssDNA on the sensor chip. This binding interaction was readily observed with the SPR.

Subsequently, the 5′-Candidate strand of DNA was flowed over the SPR sensor chip for 120 seconds and hybridization of the Candidate-bearing strand to the biotin-bearing strand effectively immobilized the Candidate on the sensor. This interaction was readily observed on the SPR.

The buffer used comprised 100 mM KCl and 10 mM Bis-Tris-Propane (BTP Buffer). Non-specific binding on the sensor was blocked by flowing a solution of BSA in BTP Buffer at pH 6 over the sensor for >2 minutes.

Separately, rubisco at varying concentrations was flowed over the sensor chip for about 3 minutes. The association of rubisco with the immobilized candidates was observed during this phase. Next, the flow of rubisco was halted and BTP Buffer alone was flowed over the chip for 5 minutes to allow observation of the dissociation of rubisco from the immobilized Candidate molecules.

Included in the design were control sensor regions wherein no Candidate molecule was present on the immobilized DNA. Non-specific binding to these controls was subtracted from the signal in the experimental regions. Once the association and dissociation of rubisco was measured at pH 6. The experiments were repeated at pH 9.

In these experiments, the on-rate (h), and off-rate (kd) of the Candidates for rubisco were measured at pH 6 and then again at pH 9. The dissociation constants (KD) were also derived. Table 8 lists the kinetic data for the binding experiments.

TABLE 8
Kinetic data for binding experiments

| | pH 6 | | | pH 9 | | |
|---|---|---|---|---|---|---|
| Candidate | Ka (1/Ms) | Kd (1/s) | KD (M) | Ka (1/Ms) | Kd (1/s) | KD (M) |
| 16 | 3.38E+03 | 2.77E−04 | 8.18E−08 | 2.71E+02 | 2.86E−03 | 9.85E−06 |
| 1 | 3.06E+03 | 3.37E−04 | 1.10E−07 | 2.85E+01 | 6.47E−04 | 2.27E−05 |

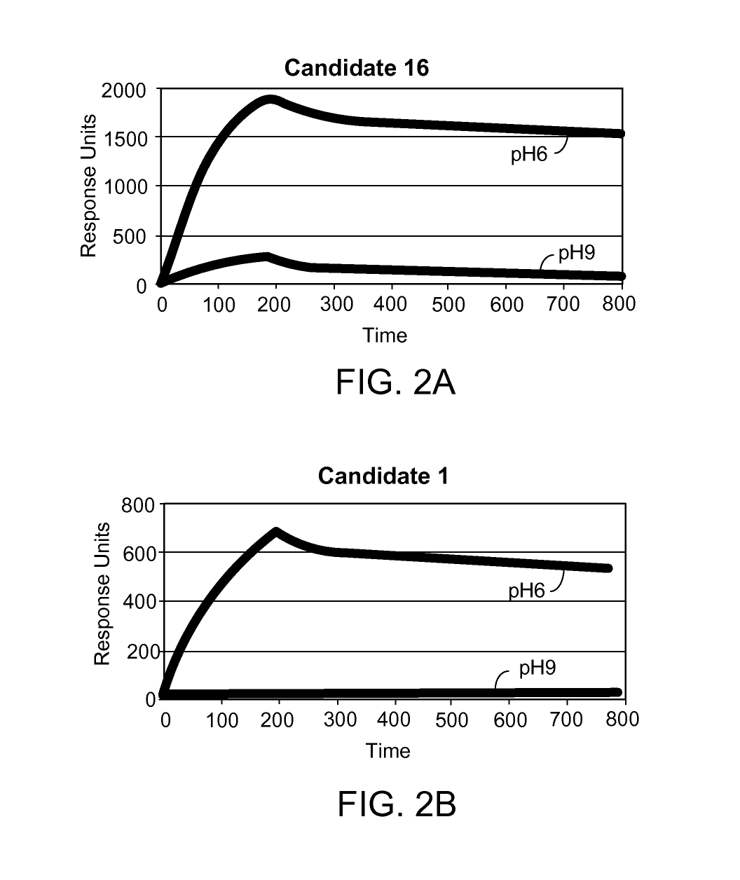

The graphs in FIGS. 2A and 2B depict the raw data used to generate the constants listed in Table 8. FIG. 2A plots SPR data for Candidate 16, while FIG. 2B plots SPR data for Candidate 1.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound comprising the structure of Formula (III): 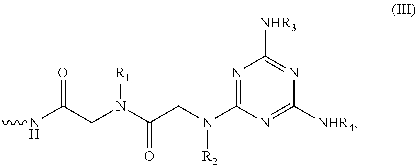
wherein the N—R1 to N—R4 groups of Formula (III) correspond, respectively, to the R1-R4 groups listed in Tables 1-5, wherein the N of each N—R group in Formula (III) is the N of an R group in the tables, and wherein the wavy line indicates the point of attachment to a substrate, optionally through a linker.

2. The compound of claim 1, wherein the compound binds a target at a first pH and does not bind the target or binds the target with a lower affinity at a second pH.

3. The compound of claim 1, wherein the N—R1 to N—R4 groups of Formula (III) correspond, respectively, to the R1-R4 groups listed in Table 2, and wherein the N of each N—R group in Formula (III) is the N of an R group in Table 2.

4. The compound of claim 3, wherein the compound binds rubisco at a pH of about 6 and releases rubisco or binds rubisco with a lower affinity at a pH of about 9.

5. The compound of claim 1, wherein the N—R1 to N—R4 groups of Formula (III) correspond, respectively, to the R1-R4 groups listed in Table 3, and wherein the N of each N—R group in Formula (III) is the N of an R group in Table 3.

6. The compound of claim 5, wherein the compound binds rubisco at a pH of about 9 and releases rubisco or binds rubisco with a lower affinity at a pH of about 6.

7. The compound of claim 1, wherein the N—R1 to N—R4 groups of Formula (III) correspond, respectively, to the R1-R4 groups listed in Table 4, and wherein the N of each N—R group in Formula (III) is the N of an R group in Table 4.

8. The compound of claim 7, wherein the compound binds leghemoglobin at a pH of about 6 and releases leghemoglobin or binds leghemoglobin with a lower affinity at a pH of about 9.

9. The compound of claim 1, wherein the N—R1 to N—R4 groups of Formula (III) correspond, respectively, to the R1-R4 groups listed in Table 5, and wherein the N of each N—R group in Formula (III) is the N of an R group in Table 5.

10. The compound of claim 9, wherein the compound binds leghemoglobin at a pH of about 9 and releases leghemoglobin or binds leghemoglobin with a lower affinity at a pH of about 6.

11. The compound of claim 1, wherein N—R1 is 3-fluoro-(2-phenyl ethanamine), N—R2 is 2-amino-N-cyclopropylacetamide, N—R3 is 2-methyl butylamine, and N—R4 is (4-bromophenyl) methanamine.

12. The compound of claim 1, wherein N—R1 is 2-methoxy-ethylamine, N—R2 is 2-(3-fluorophenyl) ethanamine, N—R3 is 4-aminobutan-1-ol, and N—R4 is 2-amino-1-phenylethanol.

13. The compound of claim 1, wherein the compound is conjugated to the substrate, optionally through a linker.

14. The compound of claim 13, wherein the substrate comprises agarose, sepharose, polystyrene, styrene, iron oxide, magnetic, or paramagnetic beads.

15. The compound of claim 14, wherein the substrate comprises sepharose beads.

16. The compound of claim 13, wherein the linker comprises a diamine.

17. The compound of claim 16, wherein the linker is 4,7,10-trioxa-1, 13-tridecanediamine.

18. The compound of claim 13, wherein the substrate is a component of a chromatography resin.

19. A method for isolating a target protein, comprising:
(a) applying a composition comprising the target protein to a substrate, wherein the substrate comprises a compound comprising the structure of Formula (III) 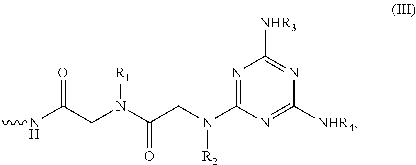
wherein the N—R1 to N—R4 groups of Formula (III) correspond, respectively, to the R1-R4 groups listed in Tables 1-5, wherein the N of each N—R group in Formula (III) is the N of an R group in the tables, and wherein the wavy line indicates the point of attachment to the remainder of the substrate, optionally through a linker;
(b) adjusting the pH of the solution to a second pH, wherein at the second pH the target protein does not bind the compound or binds the compound with lower affinity, and is eluted from the compound; and
(c) collecting the target protein that eluted from the compound.

20. The method of claim 19, wherein the compound binds a target at a first pH and does not bind the target or binds the target with a lower affinity at a second pH.

21. The method of claim 19, wherein the N—R1 to N—R4 groups of Formula (III) correspond, respectively, to the R1-R4 groups listed in Table 2, and wherein the N of each N—R group in Formula (III) is the N of an R group in Table 2.

22. The method of claim 21, wherein the compound binds rubisco at a pH of about 6 and releases rubisco or binds rubisco with a lower affinity at a pH of about 9.

23. The method of claim 19, wherein the N—R1 to N—R4 groups of Formula (III) correspond, respectively, to the R1-R4 groups listed in Table 3, and wherein the N of each N—R group in Formula (III) is the N of an R group in Table 3.

24. The method of claim 23, wherein the compound binds rubisco at a pH of about 9 and releases rubisco or binds rubisco with a lower affinity at a pH of about 6.

25. The method of claim 19, wherein the N—R1 to N—R4 groups of Formula (III) correspond, respectively, to the R1-R4 groups listed in Table 4, and wherein the N of each N—R group in Formula (III) is the N of an R group in Table 4.

26. The method of claim 25, wherein the compound binds leghemoglobin at a pH of about 6 and releases leghemoglobin or binds leghemoglobin with a lower affinity at a pH of about 9.

27. The method of claim 19, wherein the N—R1 to N—R4 groups of Formula (III) correspond, respectively, to the R1-R4 groups listed in Table 5, and wherein the N of each N—R group in Formula (III) is the N of an R group in Table 5.

28. The method of claim 27, wherein the compound binds leghemoglobin at a pH of about 9 and releases leghemoglobin or binds leghemoglobin with a lower affinity at a pH of about 6.

29. The method of claim 19, wherein N—R1 is 3-fluoro-(2-phenyl ethanamine), N—R2 is 2-amino-N-cyclopropylacetamide, N—R3 is 2-methyl butylamine, and N—R4 is (4-bromophenyl) methanamine.

30. The method of claim 19, wherein N—R1 is 2-methoxy-ethylamine, N—R2 is 2-(3-fluorophenyl) ethanamine, N—R3 is 4-aminobutan-1-ol, and N—R4 is 2-amino-1-phenylethanol.

31. The method of claim 19, wherein the compound is conjugated to the substrate, optionally through a linker.

32. The method of claim 31, wherein the substrate comprises agarose, sepharose, polystyrene, styrene, iron oxide, magnetic, or paramagnetic beads.

33. The method of claim 32, wherein the substrate comprises sepharose beads.

34. The method of claim 31, wherein the linker comprises a diamine.

35. The method of claim 34, wherein the linker is 4,7,10-trioxa-1,13-tridecanediamine.

36. The method of claim 31, wherein the substrate is a component of a chromatography resin.

37. A consumable product comprising a target protein and a compound comprising the structure of Formula (III) 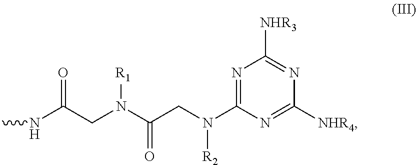
wherein the N—R1 to N—R4 groups of Formula (III) correspond, respectively, to the R1-R4 groups listed in Tables 1-5, wherein the N of each N—R group in Formula (III) is the N of an R group in the tables, and wherein the wavy line indicates the point of attachment to a substrate, optionally through a linker.

38. The consumable product of claim 37, wherein the compound is present at a concentration of less than 1,000 parts per million target protein.

39. The consumable product of claim 37, wherein the consumable product is substantially free of chlorophylls, chlorins, metalloids, transition metals, celluloses, or complex polysaccharides.

40. The consumable product of claim 37, wherein the consumable product is food-safe.
